(19) United States
(12) Patent Application Publication
(10) Pub. No.: US 2012/0083593 A1
(43) Pub. Date: Apr. 5, 2012

(54) Separation and Purification of Stevioside and Rebaudioside A

(75) Inventors: Jian Liu, Scarborough (CA); Kai Zhang, Shanghai (CN); Shen Guo, Shanghai (CN)

(73) Assignee: SHANGHAI YONGYOU BIOSCIENCE INC., Shanghai (CN)

(21) Appl. No.: 13/249,773

(22) Filed: Sep. 30, 2011

Related U.S. Application Data
(60) Provisional application No. 61/388,691, filed on Oct. 1, 2010.

(51) Int. Cl. C07H 15/24

(52) U.S. Cl. 536/18.1

(57) ABSTRACT

A commercially viable method is provided herein for isolating and purifying steviol glycosides from a source containing the steviol glycosides. The method includes the first step of passing an organic solution containing the steviol glycosides through a chromatographic column, where the packing medium in the column has been compressed substantially to avoid voids therein, and then the packing medium is maintained under a pressure of up to about 1500 psi to thereby provide an organic solution containing impure stevioside derivatives.

### CROSS REFERENCE TO RELATED APPLICATION

This application claims priority to U.S. Application No. 61/388,691, filed Oct. 1, 2010, which is incorporated herein by reference in its entirety.

### TECHNICAL FIELD

This description relates to a method for the recovery of purified individual stevioside and rebaudioside A from the stevia rebaudiana Bertoni plant.

### BACKGROUND

The worldwide demand for high potency sweeteners is increasing and the demand for alternative sweeteners is expected to increase.

The desire for low calorie, or no-calorie, sweeteners led originally to the use of artificial sweeteners, e.g., first saccharin and then the cyclamates and aspartame, as substitutes for sucrose. Artificial sweeteners were developed for use by diabetics and to decrease the calorie content of food preparations, especially for a low-calorie diet. These sweeteners are frequently sweeter than natural sugar and may, in order to achieve the same measure of sweetening action, be used in small amounts.

### SUMMARY

By a first broad aspect, a method is performed for isolating and purifying steviol glycosides from a source containing steviol glycosides. The method includes passing an organic solution containing steviol glycosides through a chromatographic column, where the packing medium in the column has been compressed substantially to avoid voids therein, and then maintaining the packing medium under a pressure of up to about 1500 psi, the high pressure being used to load the packing medium as a slurry into the column. The eluting is done under medium pressure, e.g., less than 300 psi.

By a second broad aspect, the method includes passing an organic solution containing steviol glycosides through a first chromatographic column, where the packing medium in the column has been compressed substantially to avoid voids therein, and then maintaining the packing medium under a pressure of up to about 1500 psi thereby to provide an organic solution containing impure stevioside derivatives; and then passing the organic solution containing impure stevioside derivatives through a second chromatographic column in which the packing medium in the column has been compressed to obviate voids in said column, and then maintaining the packing medium at a pressure of up to about 400 psi.

By a third broad aspect, the method includes passing an organic solution containing stevioside derivatives through a first chromatographic column, where the packing medium in the column has been compressed substantially to avoid voids therein, maintaining the packing medium under a pressure of up to about 1500 psi, thereby to provide an organic solution containing impure stevioside derivatives; passing an organic solution containing extracted impure stevioside derivatives through a second chromatographic column in which the packing medium in the column has been compressed to obviate voids in said column and then maintaining the packing medium at a pressure of up to about 400 psi, thereby to provide a syrup of extracted stevioside and rebaudioside A in an organic solvent; and the third step of passing the syrup of said extracted stevioside and rebaudioside A in an organic solvent through a chromatographic column in which a packing medium is compressed to obviate voids in said column, and maintaining the packing medium at a pressure of up to about 100 psi.

By a fourth broad aspect, the method includes extracting a source of steviol glycoside in an organic extractant; coating the extractant with an absorbent agent; loading the so-coated steviol glycoside in a column containing an absorbent agent; eluting, with an organic solvent mixture at a pressure of between 30 and 80 psi to generate fractions containing stevioside and rebaudioside A compounds; and, crystallizing the fractions to provide a mother liquor containing stevioside and rebaudioside; and finally, concentrating the mother liquor, to obtain and recover substantially-pure stevioside (>98%) and rebaudioside A (>98%) compound.

By a fifth broad aspect, the method includes combining crude stevioside or rebaudioside A and an organic solvent or an aqueous organic solvent to form stevioside or rebaudioside A solution, the aqueous organic solution comprising water in an amount from about 5% to about 12% by weight; and crystallizing from the stevioside or rebaudioside A solution in a single step.

By a sixth broad aspect, of the method includes dissolving crude steviol glycosides in a water soluble organic solvent; coating the dissolved steviol glycosides with a NH(CH₂)₂NH₂ functionalized polymethacrylate/DVB co-polymer resin; evaporating the water soluble organic solvent to provide a coated steviol glycoside material; loading the so-dried coated steviol glycosides material into the top of a medium or low pressure preparative chromatography column packed with a NH(CH₂)₂NH₂ functionalized polymethacrylate/DVB co-polymer; eluting the steviol glycosides with a step gradient methanol:acetone mixture; to elute a mixture of rebaudioside A and stevioside; and forming and recovering crystals comprising both substantially pure rebaudioside A and substantially pure stevioside.

By a seventh broad aspect, a substantially pure stevioside and rebaudioside A composition is provided comprising rebaudioside A in a purity greater than about 99% by weight on a dry basis, and stevioside in a purity greater than 98% by weight on a dry basis, which has been crystallized from a rebaudioside A solution in a single step.

The separation is simple, fast and provides higher purity and higher yield than the traditional recrystallization or other purification methods that extract either rebaudioside A or stevioside, but do not obtain two products in one process. The method described herein provides for the extraction and purification of both rebaudioside A and stevioside from a steviol glycosides source material (e.g., stevia rebaudiana Bertoni plant) in a single chromatography column step and by providing a way to extract both products (rebaudioside A and stevioside) in one method step, makes the production cost much less and makes more sense economically. The composition described herein provides a non-sucrose sweetener that is of natural origin.

### DESCRIPTION OF THE DRAWINGS

The drawings illustrate several exemplary implementations of the above-described method, in which:

FIG. 1 is a chromatogram of an HPLC separation of steviosides.

FIG. 2 is a chromatogram of a crude rebaudioside A crystal from crystallization of Experiment 3.

FIG. 3 is a chromatogram of a stevioside crystal from crystallization of Experiment 5.

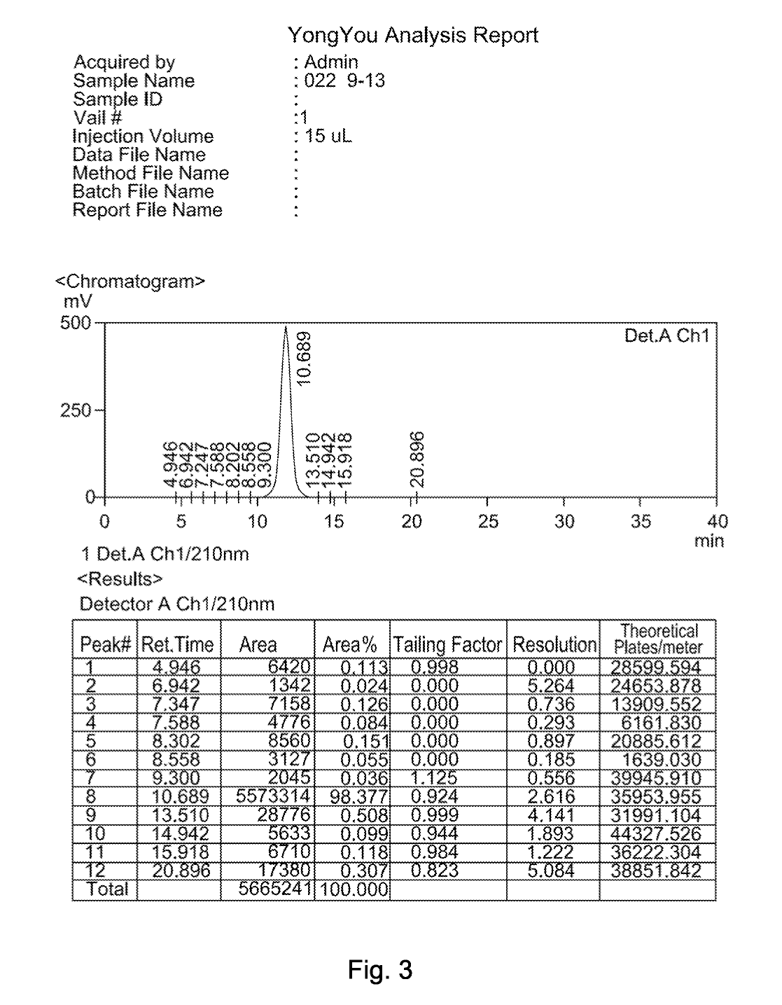

FIG. 4 is a chromatogram of a rebaudioside A crystal from crystallization of Experiment 5.

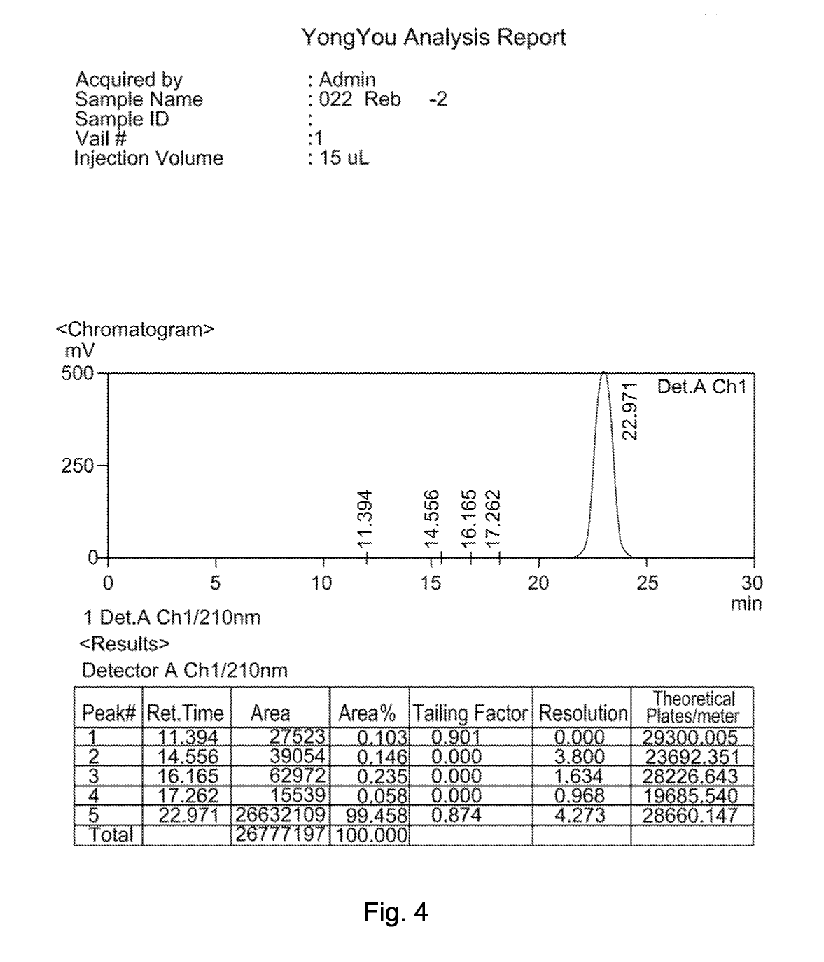

### DETAILED DESCRIPTION

Stevia rebaudiana Bertoni is cultivated in China, Korea, Taiwan, Thailand, Malaysia, Saint Kitts & Nevis, Brazil, Colombia, Peru, Uruguay, Philippines, and Israel and is a plant native to Paraguay. This plant per se has been used as a sweetening agent. The leaves of this plant have been reported to contain stevioside, rebaudiosides A and rebaudiosides C (dulcoside C). These compounds are alleged to be present in the largest quantities and are said to the sweetest. Efforts have been made to recover and separate the sweetest components for commercial use as sweeteners.

Steviol glycosides derived from stevia rebaudiana Bertoni are presently being used as serviceable artificial sweeteners and are added to low-calorie foods or as replacement for natural sugar, since they have shown no disadvantageous effects in clinical trials. In addition to the advantage of being natural plant products, steviol glycosides have functional and sensory properties superior to those of many high potency sweeteners.

Stevia glycosides extracted from the leaves and stems of stevia rebaudiana Bertoni have an intense sweetness substantially greater than ordinary sugar (sucrose), yet are low in calories. They have been used as natural sweeteners for over 20 years in Asia and are also approved as a food additive in Canada and the United States. In addition, they are approved, and are available commercially, in China, Hong Kong, Indonesia, Viet Nam, France, Switzerland, the Russian Federation, Turkey, Argentina, Brazil, Chile, Mexico, Paraguay, Peru, Australia, and New Zealand. Thus there is a high commercial demand for steviol glycosides and for their cost effective preparation and purification methods.

Stevioside and rebaudioside A have had their physical and sensory properties well characterized. Stevioside and rebaudioside A were tested for stability in carbonated beverages and found to be both heat and pH stable. Stevioside is between 110 and 270 times sweeter than sucrose, and rebaudioside A is between 150 and 320 times sweeter than sucrose. Both stevioside and rebaudioside A are synergistic in mixtures with other high potency sweeteners, e.g., aspartame, and are good candidates for inclusion in blends.

Processes for preparing high purity rebaudioside A include high-performance liquid chromatography, thin layer chromatography, drop counter-current chromatography, capillary electrophoresis, and supercritical fluid extraction. Due to small handling capacity and high cost, these processes may not suitable for industrial production.

Among the methods for extracting stevioside and rebaudioside from stevia rebaudiana Bertoni are the following:

High-performance liquid chromatography; thin layer chromatography; drop counter-current chromatography; capillary electrophoresis; supercritical fluid extraction, dissolving stevia glycosides in ethanol, subjecting that solution to solid-liquid separation, decolorizing and drying; dissolving crude stevia glycosides in an ethanol-water solvent, followed by filtering, desalinating the filtrate with ion exchange resin, decolorizing with active charcoal, and evaporation; separating enriched rebaudioside A through selective adsorption through macroporous adsorption resins followed by recrystallization; a method for purifying stevia glycosides through a liquid-liquid extraction of crude stevia glycosides solution extraction using fatty alcohol or fatty alcohol plus diluent, after which water is used for reverse extraction; a process of extracting stevia glycosides through resins, by soaking the dried stevia plant leaves, plate filtration, adsorption, desalination, decolorization with ordinary strongly alkaline resin, concentration and drying; a method to manufacture high-content rebaudioside A using water or aqueous solvent to extract stevia glycosides from the dried stems or leaves of stevia plant, and then separation and collection, to provide an extract of rebaudioside; the separation of two of the stevia rebaudiana Bertoni materials, stevioside and rebaudioside A, with a high-performance liquid chromatography (HPLC) system; methods for the extraction and purification of steviosides using organic solvents, e.g., methanol, ethanol or ether, followed by adsorption of the steviosides first on a resin with subsequent elution with an organic solvent; extractions of leaves, roots or fruits of natural plants in the form of liquid or solid; by gas-chromatography by converting the natural sweet compounds to the corresponding volatile compound by trimethylsilylation or by Smith decomposition; or by thin layer chromatography with a long time for development separation.

A typical profile for the four major glycosides found in the leaves of Stevia comprises 0.3% dulcoside, 0.6% rebaudioside C, 3.8% rebaudioside A, and 9.1% stevioside. Dulcoside and rebaudioside C are present in small quantities but are the components in stevia rebaudiana Bertoni extract that give a bitter aftertaste, and it is therefore desirable to remove them from the stevia rebaudiana Bertoni extract.

Ordinary liquid chromatography imposes trade-offs between the factors of purity of product and quantity of product purified in a given time: the higher the purity the less the quantity of product purified per unit of time. It was proposed to substitute very large columns by high performance liquid chromatography (HPLC) equipment, which uses high fluid pressures to drive eluting solvent continuously through very tightly-packed high surface area packing Since many other stevia glycosides have very similar chemical structures as they all share the same diterpene skeleton and differ only slightly in the types, quantities, and structural patterns of glycoside moieties, it is still difficult to obtain high purity rebaudioside A at an industrial scale.

Impure ordinary stevia glycosides have certain drawbacks, one of which is a prolonged aftertaste. On the other hand, rebaudioside A does not possess a substantial aftertaste and has a sweetness flavor comparable to sucrose.

### EXAMPLES

The method described herein relates to the separation and purification of stevioside and rebaudioside A from a steviol glycoside source material, which is extracted from stevia rebaudiana Bertoni.

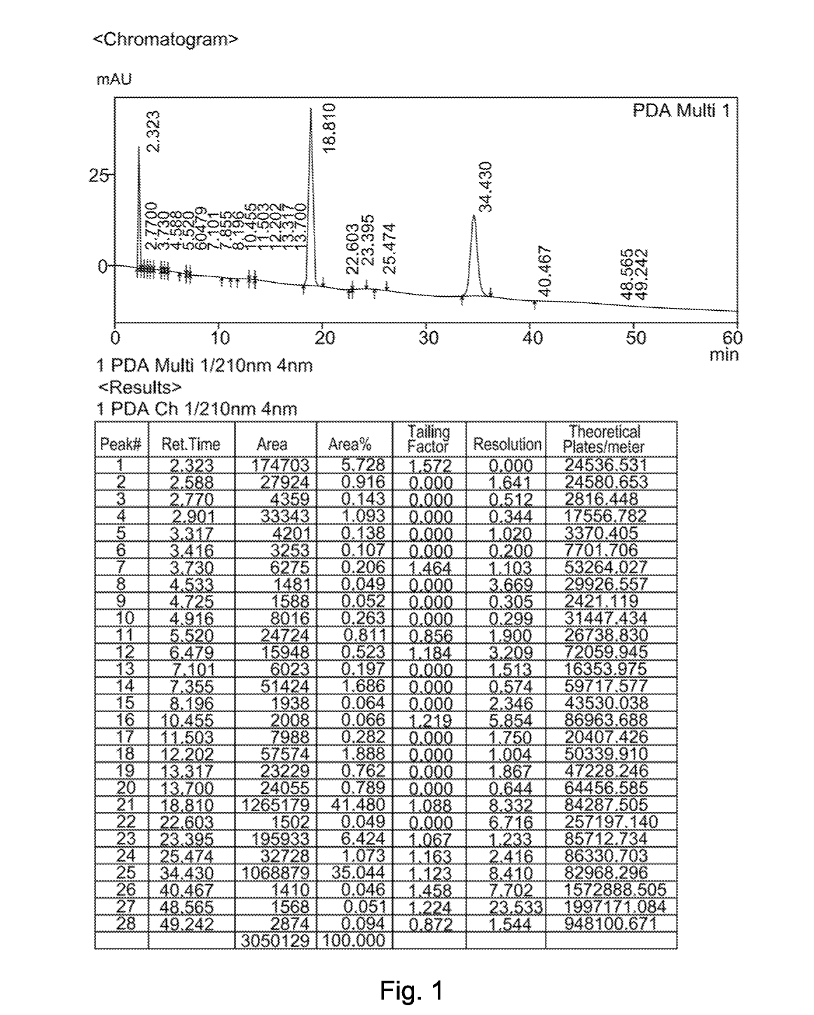

Referring to FIG. 1, the source material contains approximately 41.5% of stevioside and 35.0% of rebaudioside A by HPLC analysis. The separation of the stevioside and the rebaudioside A from this source material is achieved by using a medium or low pressure preparative chromatography column packed with a suitable resin, namely a polystyrene-DVB resin (e.g., that sold by Zhejiang Zhenggunang Industrial Company; or under the name PROTEOMIX™ sold by Tosoh Bioscience LTD) or a polymethacrylate resin, (e.g. Plexiglass™) or a polyaromatic resin (e.g., a polystyrene resin cross-linked with divinyl benzene sold under the name POLYPACK-2™ by Hewlett-Packard F & M Scientific division; or by Amberlite resin Ltd, or AMBERLITE™ sold by Rohm & Haas, or DIAION™ sold by Mitsubishi Chemical Corporation or DOWEX™ sold by Dow chemical) or a functionalized polymethacrylate-DVB resin, (TSK-GEL™ sold by Tosoh Bioscience LTD) or a functionalized polystyrene-DVB resin, or an amino (NH₂) bonded polymethacrylate/DVB co-polymer resin.

### Experiment 1

Commercially available crude steviol glycosides material was purchased from Jiuqian, Ganshu, People's Republic of China. The crude material was analyzed by HPLC and was a mixture of 41% stevioside and 35.8% rebaudioside A plus other impurities.

The chromatography column was: 1.8×40 cm, loaded with 80 ml of a NH(CH₂)₂NH₂ functionalized polymethacrylate/DVB co-polymer absorbent resin. Two grams of crude steviol glycoside was dissolved in 20 ml ethanol, then coated with 4 ml of the above-identified absorbent resin and the solvent was then evaporated at vacuum through a rotary evaporator.

The so-coated steviol glycoside was loaded on to the top of the chromatographic column and was eluted with a step-gradient solvent mixture (methanol:acetone), starting from 10% methanol in acetone, 12% methanol in acetone, then 15% methanol in acetone and ending at 20% methanol in acetone at a rate of 4-5 ml/min.

Fractions were collected at 40 ml/each, and checked by HPLC. The fractions containing stevioside and rebaudioside A were combined. After combination of the fractions and reduction on a rotary evaporator, needle-like crystals were formed in the container. The crystals were filtered out and dried under vacuum (60° C.), and the purity checked by HPLC. The purity was 98.34% for stevioside and 98.68% for rebaudioside A.

The yield was as follows: stevioside 0.78 g (95% recovery based on dried crude material); and rebaudioside A 0.65 g (92% recovery based on dried crude material).

### Experiment 2

The chromatography column was: 16×50 cm, loaded with 80 ml of NH(CH₂)₂NH₂ functionalized polymethacrylate/DVB co-polymer absorbent resin. 400 g of crude steviol glycoside was dissolved into 1200 ml of ethanol, coated with 600 g of Celite 545™. The solvent was evaporated under vacuum through a rotary evaporator. The coated material was loaded onto the top of the chromatographic column and eluted with a step-gradient solvent mixture (methanol:acetone). The gradient was from 5% methanol to 40% methanol in acetone. The total solvents usage was 100 liters, including 2 bed volumes (BV) of 5%; 5 BV 10%; 2 BV 12%; 1.5 BV 15%; and then 2 BV 40% at a flow rate of 250 ml/min.

Stevioside was collected at the 10%-12% methanol fractions and was crystallized from those fractions directly before any concentration. The crystalline product was filtrated out and checked by HPLC, which showed a purity of 98.01%. The liquid was concentrated to dryness, the white solid was dissolved in a small amount of methanol, and 4 volumes of acetone were added. The mixture was kept at room temperature overnight. A white crystal was collected and dried. A total 154.2 grams of stevioside as a white crystalline powder was obtained, the recovery being a yield of 94%. The column was further washed with 15% methanol in acetone and then 40% methanol in acetone. The fractions were combined and concentrated to dryness, the residue was dissolved in methanol and some acetone was added. The mixture was kept at room temperature overnight. The white crystals (approximately 125 g) were filtered out and dried. The purity of the rebaudioside A product was 98.4% and the yield was 125 g (93% recovery).

### Experiment 3

1,100 grams of crude steviol glycoside was dissolved into 2,000 ml of 50% methanol, stirred for 20 minutes at 60° C., then 12,000 ml of acetone was added and the mixture was stirred for another 10 minutes, then kept at room temperature for 24 hours. Rebaudioside A was formed as a needle-like crystal and was filtered out and dried at 60° C.

In general, high performance liquid chromatography (HPLC) is used to determine the purity of the final products manufactured by the methods described herein. In the generalized HPLC analysis method used, an analysis sample is created by dissolving 0.1 g of dried sample in 25 ml of water. The HPLC apparatus comprises a Shimadzu LC-1 OA system with a Shimadzu SPD-1 OA variable wavelength detector. A 250 mm×4.6 mm i.d. 5 μm NH₂ column was used with an isocratic mobile phase consisting of 80% acetonitrile/20% water and a flow rate of 1.0 ml/min. Peaks were detected at 210 nm. Stevioside and rebaudioside A were quantified by comparison with a standard sample.

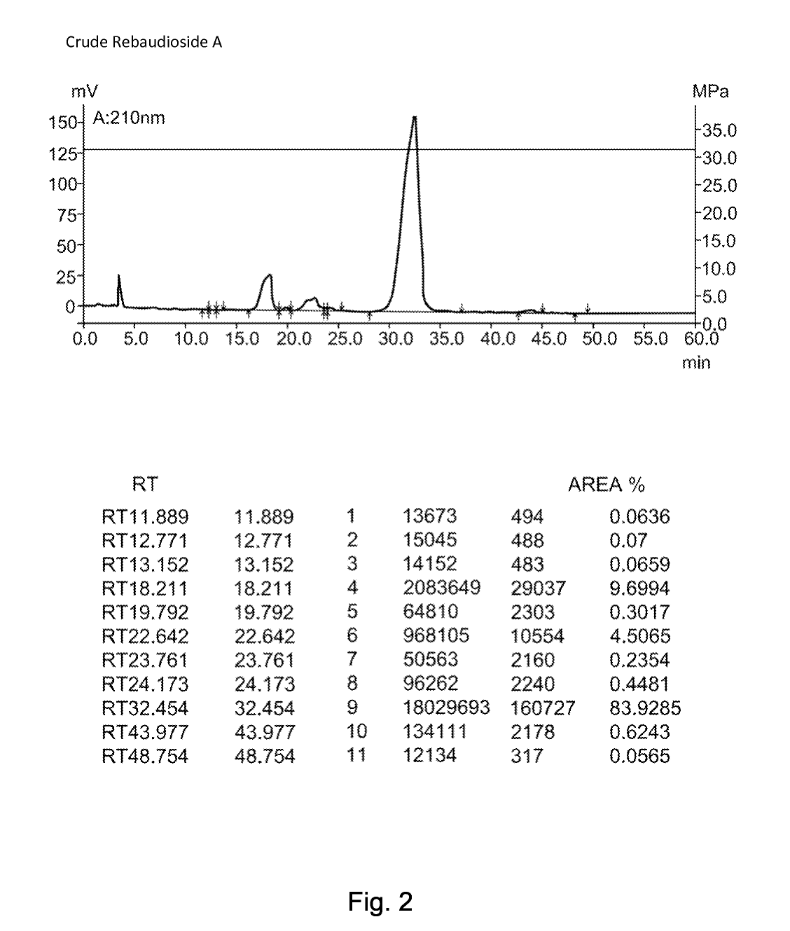

The purity of the rebaudioside A so extracted was checked by HPLC. The analysis showed that the crystalline product had a purity of 83.7% (380 g, as seen in FIG. 2). This white crystal-like product was dissolved in 800 ml of ethanol and coated with 800 ml of polystyrene/DVB co-polymer resin and then dried at 60° C. under vacuum in a rotary evaporator to yield a loading material that is ready for column purification (as described below as Purification of Preparation 1). After separating rebaudioside A, the mother liquid was concentrated to dryness at 60° C. under vacuum in a rotary evaporator, and the residue, which contains 73% stevioside, was re-dissolved in ethanol, then coated to 1,000 ml polystyrene/DVB resin. The mixture was dried in a rotary evaporator at 70° C. under vacuum to yield a loading material (as described below as Purification of Preparation 2) that is ready for column purification.

### Purification of Preparation 1:

The crude material (produced by Experiment 1) was loaded on to the top of an 8 liter, low pressure, preparative chromatography column packed with NH(CH₂)₂NH₂ functionalized polymethacrylate/DVB co-polymer absorbent resin, eluted with 15% methanol in acetone for 2 bed volumes, then eluted with 30% methanol in acetone to yield fractions that contain rebaudioside A and stevioside.

The fractions containing stevioside were collected and combined, then concentrated to dryness, the residue was dissolved in hot methanol, and then kept at room temperature overnight. The resultant needle-like crystals were filtered out and dried in a vacuum oven to yield 37 g of stevioside as a white crystalline powder, whose purity was >99%.

The fractions containing rebaudioside A were collected and concentrated to dryness in a rotary evaporator at 50° C. under vacuum. The residue was then dissolved in a small amount of hot methanol, and acetone was added to the methanol solution to achieve a ratio of methanol to acetone of 15:85. The mixture was kept at room temperature overnight. The resultant needle-like white crystals were filtered out and dried in a vacuum oven to yield 302 grams of rebaudioside A as a white crystalline powder, whose purity was >98%.

### Purification of Preparation 2

The crude material (produced by Experiment 2) was loaded onto the top of an 8 liter low pressure preparative chromatography column packed with NH(CH₂)₂NH₂ functionalized polymethacrylate/DVB co-polymer absorbent resin, eluted with 10% methanol in acetone for 2 bed volumes, then eluted with 14% methanol in acetone to yield fractions that contain stevioside. After stevioside was washed out, the column was eluted with 20% methanol in acetone to yield fractions containing rebaudioside A and stevioside.

The fractions containing stevioside were collected and combined, then concentrated to dryness. The residue was then dissolved in hot methanol and then kept at room temperature overnight. The resultant needle-like crystals were filtered out and dried in a vacuum oven to yield 375 g of stevioside as a white crystalline powder whose purity was >99%.

The fractions containing rebaudioside A were collected and concentrated to dryness in a rotary evaporator at 50° C. under vacuum. The residue was then dissolved in a small amount of hot methanol, and acetone was added to the methanol solution (the ratio of methanol to acetone is 15:85). The mixture was kept at room temperature overnight, and the needle like crystals that formed were collected through filtration. The crystals were then dried in a vacuum oven to yield 55 grams of rebaudioside A whose purity was 98.4%.

The products rebaudioside A and stevioside from the above mentioned purification methods were combined. The total recovery yield for rebaudioside A in this process is 357 grams or 92.5% and for stevioside is 412 grams or 91.3%.

### Experiment 4

### Preparation (LPLC)

The column was 300×1000 mm. 60 liters of polystyrene/DVB macroporous polymer resin absorbent was loaded into the chromatographic column. 4,000 g of the crude steviosides to be purified was dissolved into 7,000 ml of 50% methanol and coated with the polystyrene/DVB macroporous absorbent polymer resin. 15 liters of acetone:water wash solvent was used, starting from 5% water to 40% water in acetone. The total solvents usage was 540 liters, at a flow rate of 1,000 ml/min.

Stevioside was collected at the 8%-10% water fractions and was crystallized from the fractions directly before any concentration. The crystalline product was filtrated out and checked by HPLC which showed a purity of >96%.

The liquid was concentrated to dryness under vacuum. The syrup was dissolved in a small amount of water and 7 volumes of methanol were added. The mixture was kept at room temperature overnight. White crystals were collected and dried. A total of 1,440 grams of white crystalline powder was obtained. The white crystalline powder was added into a 20 liter glass reactor. Then 3 liters of 50% methanol was added. The mixture was stirred at 60° C. until the solid was completely dissolved and then 7 liters of pure methanol was added. The mixture was cooled to room temperature. A large amount of white crystal was formed which was filtered out and dried under vacuum to yield 1,101 grams of stevioside with a purity of >98%.

The column was continued to be washed by 10% water in acetone and then with 40% water in acetone. The fractions which contained rebaudioside A were combined and concentrated to dryness. The residue was dissolved in 700 ml of water and 2 liters of ethanol was added. The mixture was heated at 65° C. for 30 minutes. Then 5 liters of ethanol were added and the mixture was then poured into a stainless container which was kept at room temperature overnight. The needle-like crystals which formed were filtered out and dried under vacuum to yield 986 grams rebaudioside A as white crystalline powder which had a purity of 98.4%.

### Experiment 5

### INDUSTRIAL SCALE LIQUID CHROMATOGRAPHY

The column was 700×3,000 mm and was packed with 1,100 liters of polystyrene/DVB macroporous absorbent resin. 70 kg of the crude steviosides to be purified was dissolved in 150 L of water and was coated with 200 liters of polystyrene/DVB macroporous absorbent resin. The coated crude steviosides were dried under vacuum in a rotary evaporator at 80° C. for 5 hours. Finally the coated material had a moisture content of 15%.

10,000 liters of acetone:water wash solvent was used, starting from 5% water to 40% water in acetone. The total solvents usage was 10,000 liters at a flow rate of 16 L/min. Stevioside was collected at the 8%-10% water fractions and was concentrated in a rotary spray evaporator at 40° C. under vacuum to yield a concentrated syrup. The syrup was dissolved in one equivalent of water at 70° C. and then 7 equivalents of methanol were added. The mixture was kept at room temperature overnight. A large amount of white crystals was formed which was filtered out and dried under vacuum to yield 21 kg of stevioside with a purity of >98%.

The column was continued to be washed with 10% water in acetone and then with 40% water in acetone. The fractions which contain rebaudioside A were combined and concentrated to dryness. The residue was dissolved in one equivalent of water and then 8 equivalents of ethanol was added, the mixture was heated at 65° C. for 30 minutes, then kept at room temperature overnight. The needle-like crystal was filtered out and dried under vacuum to yield 20 kg of rebaudioside A as a white crystalline powder which has a purity of >99%.

The commercially-useful recovered and purified individual rebaudioside A and stevioside from stevia rebaudiana Bertoni is provided in commercially-useful quantities in a simple and cost effective manner.

What is claimed is:

1. A method for isolating and purifying steviol glycosides from a source containing said steviol glycosides, the method comprising:
loading a steviol glycoside, which has been coated with an absorbent resin, onto a chromatographic column, which has been loaded with a slurry of the absorbent resin, and then subjecting the column to a high pressure that is less than or equal to about 1500 psi and is sufficient to compress that absorbent resin to avoid voids therein; and
eluting an organic solution through the loaded chromatographic column under a medium pressure that is less than about 300 psi to thereby provide an organic solution containing stevioside and rebaudioside A.

2. The method of claim 1, wherein eluting the organic solution under the medium pressure comprises eluting the organic solution under a pressure that is between about 70 and about 200 psi.

3. The method of claim 1, wherein the absorbent resin is selected from the group consisting of: a polystyrene-DVB co-polymer resin; a polymethacrylate resin; a polyaromatic resin; an amino (NH₂) bonded polymethacrylate/DVB co-polymer resin; a functionalized polymethacrylate-DVB resin; a functionalized polystyrene-DVB resin; and a polyaromatic resin.

4. The method of claim 1, wherein eluting the organic solution comprises eluting a step gradient organic solution that is selected from the group consisting of: acetone/water, methanol/water; acetonitrile/water; isopropanol/water; and a mixture of methanol, ethanol and water.

5. The method of claim 1, wherein eluting the organic solution through the loaded column comprises eluting at a flow rate of about 1 BV/hour.

6. The method of claim 1, further comprising crystallizing impure steviol glycosides from the organic solution containing the stevioside and rebaudioside A.

7. The method of claim 6, further comprising recovering substantially pure stevioside and rebaudioside A from the organic solution containing stevioside and rebaudioside A.

8. The method of claim 1, further comprising removing impurities from the steviol glycoside prior to coating the steviol glycoside with the absorbent resin.

9. The method of claim 8, wherein removing impurities comprises removing lignin and flavonoid impurities from the steviol glycoside.

10. The method of claim 1, wherein the steviol glycoside that is coated comprises a solution of crude crystalline stevioside and rebaudioside A.

11. The method of claim 1, wherein the steviol glycoside that is coated comprises at least stevioside, rebaudioside A, and dulcoside.

12. The method of claim 1, wherein the source of the steviol glycosides comprises a plant source.

13. The method of claim 1, wherein the steviol glycoside that is coated comprises one or more of rebaudioside A and stevioside.

14. A method for isolating and purifying steviol glycosides from a source containing steviol glycosides, the method comprising:
coating steviol glycoside with a suitable absorbent agent;
loading the coated steviol glycoside into a column, wherein the column contains a suitable absorbent agent;
eluting an organic solution containing the steviol glycosides through the chromatographic column, wherein the suitable absorbent agent in the column has been compressed substantially to avoid voids therein; and
maintaining the column under a high pressure of up to about 1500 psi, the high pressure being used to load the packing medium as a slurry into the column;
wherein eluting is done under a medium pressure of less than about 300 psi to thereby provide an organic solution containing impure stevioside derivatives.

15. The method of claim 14, further comprising:
passing the organic solution containing impure stevioside derivatives through a chromatographic column in which a packing media in the column has been compressed to obviate voids in the column; and
maintaining the column at a pressure of up to about 400 psi, to thereby provide an extracted stevioside and rebaudioside A in an organic solvent.

16. The method of claim 15, further comprising:
passing a syrup of the extracted stevioside and rebaudioside A in an organic solvent through a chromatographic column in which a packing medium is compressed to obviate voids in the column; and
maintaining the column at a pressure of up to about 100 psi.

17. The method of claim 14, wherein the suitable absorbent agent is selected from the group consisting of: a polystyrene-DVB co-polymer resin; a polymethacrylate resin; a polyaromatic resin; an amino (NH₂) bonded polymethacrylate/DVB co-polymer resin; a functionalized polymethacrylate-DVB resin; a functionalized polystyrene-DVB resin; and a polyaromatic resin.

18. A method of isolating and purifying stevioside analogues from a source containing steviol glycoside, the method comprising:
extracting a source of steviol glycoside in an organic extractant;
coating the steviol glycoside in an organic extractant with a suitable absorbent agent;
loading the coated steviol glycoside into a chromatographic column, wherein the column contains a suitable absorbent agent;
eluting an organic solvent mixture through the column at a pressure of between about 30 and about 80 psi to generate fractions containing stevioside and rebaudioside A compounds;
crystallizing the fractions to provide stevioside and rebaudioside A and a mother liquor; and
concentrating the mother liquor to provide pure stevioside (>98%) and rebaudioside A (>98%).

19. A method for purifying stevioside and rebaudioside A, the method comprising:
combining crude stevioside or rebaudioside A and an organic solvent or an aqueous organic solvent to form a stevioside or rebaudioside A solution, the aqueous organic solution comprising water in an amount from about 5% to about 12% by weight;
crystallizing from the stevioside or rebaudioside A solution in a single step a substantially pure stevioside and rebaudioside A composition comprising stevioside in a purity greater than 98% by weight on a dry basis; and
crystallizing from the rebaudioside A solution in a single step a substantially pure rebaudioside A composition comprising rebaudioside A in a purity greater than about 99% by weight on a dry basis.

20. A substantially pure stevioside and rebaudioside A composition comprising substantially-pure stevioside in a purity greater than 98% by weight on a dry basis, and substantially pure rebaudioside A in a purity greater than about 99% by weight on a dry basis.
